(19) United States
(12) Patent Application Publication
(10) Pub. No.: US 2026/0090857 A1
(43) Pub. Date: Apr. 2, 2026

(54) MEDICAL LIGHTING DEVICE

(71) Applicant: Joonheon KIM, Chuncheon-si (KR)

(72) Inventors: Joonheon KIM, Chuncheon-si (KR); Won Suk CHOI, Seongnam-si (KR)

(73) Assignee: Joonheon KIM, Chuncheon-si (KR)

(21) Appl. No.: 19/411,073

(22) Filed: Dec. 5, 2025

Related U.S. Application Data
(63) Continuation of application No. PCT/KR2024/007736, filed on Jun. 5, 2024.

(30) Foreign Application Priority Data
Jun. 7, 2023 (KR) 10-2023-0073114

(51) Int. Cl. A61B 90/35; A61B 90/50

(52) U.S. Cl. CPC A61B 90/35; A61B 2090/502

(57) ABSTRACT

According to one embodiment of the present invention, provided is a medical lighting device comprising: a frame; and a light source device including a light arm coupled to one end of the frame and a plurality of lamp modules coupled to the light arm, wherein the light source device further includes angle adjusting devices that connect the light arm and the plurality of lamp modules so the angle of each of the plurality of lamp modules with respect to the light arm is adjustable.

### CROSS-REFERENCE TO RELATED APPLICATIONS

This application is a continuation of International Application No. PCT/KR2024/007736 filed on June 5, 2024, which claims priority to Korean Patent Application No. 10-2023-0073114 filed on June 7, 2023, the entire contents of which are herein incorporated by reference.

### TECHNICAL FIELD

Embodiments of the disclosure relate to a medical lighting device.

### BACKGROUND ART

Headlamps are typically used in, for example, mining, medical, and especially surgical fields, attached to headbands, caps, or helmets. These headlamps may advantageously illuminate the actual fields of view of wearers without the need for fixed floodlights or portable lamps.

Among these headlamps, there are headlamps each of which is provided with a plurality of light sources to achieve a shadowless effect without obstructing the field of view of the user. However, because each conventional headlamp has a fixed light source device connected to the frame, the angle of each light source is non-adjustable, and the light source device is provided as a single integrated assembly.

### DISCLOSURE OF INVENTION

### Technical Problem

An objective of the disclosure is to provide a medical lighting device which includes a plurality of light sources to ensure the field of view of a user, and facilitates adjustment of the angles of the light sources and enables disassembly and assembly of respective lamp modules of a light source device, thereby improving the convenience of use.

### Solution to Problem

An embodiment of the disclosure provides a medical lighting device including: a frame; and a light source device including a light arm which is coupled to one end of the frame and a plurality of lamp modules which are coupled to the light arm, wherein the light source device further includes angle adjustment devices each of which connects the light arm and a corresponding lamp module of the plurality of lamp modules so that an angle of the lamp module is adjustable relative to the light arm.

### Advantageous Effects of Invention

The medical lighting device according to embodiments of the disclosure may ensure the field of view of a user, facilitate adjustment of the angles of the light sources, and enable disassembly and assembly of the light sources.

### BRIEF DESCRIPTION OF DRAWINGS

FIG. 1 is a perspective view illustrating a medical lighting device according to an embodiment of the disclosure.

FIG. 2 is a perspective view only illustrating a light source device in the medical lighting device according to an embodiment of the disclosure.

FIG. 3 is an exploded perspective view of the light source device shown in FIG. 2.

FIG. 4 is an exploded perspective view of an angle adjustment device of the light source device shown in FIG. 3.

FIG. 5 is a cross-sectional view illustrating a coupling structure between a fixed link member and a light arm in the medical lighting device according to an embodiment of the disclosure.

FIG. 6 is a view illustrating a disassembly/assembly structure and a power supply structure for lamp modules and a light arm in the medical lighting device according to an embodiment of the disclosure.

FIG. 7 is a perspective view only illustrating a light source device in a medical lighting device according to another embodiment of the disclosure.

FIG. 8 is a perspective view illustrating the states of respective lamp modules in the light source device shown in FIG. 7 before and after angle adjustment.

FIG. 9 is a side view illustrating the states of respective lamp modules in the light source device shown in FIG. 7 before and after angle adjustment.

FIG. 10 is an exploded perspective view illustrating the light source device shown in FIG. 7.

FIG. 11 is an exploded perspective view illustrating an angle adjustment device in the light source device shown in FIG. 7.

### BEST MODE FOR CARRYING OUT THE INVENTION

An embodiment of the disclosure provides a medical lighting device including: a frame; and a light source device including a light arm which is coupled to one end of the frame and a plurality of lamp modules which are coupled to the light arm,

wherein the light source device further includes angle adjustment devices each of which connects the light arm and a corresponding lamp module of the plurality of lamp modules so that an angle of the lamp module is adjustable relative to the light arm.

In an embodiment of the disclosure, each of the angle adjustment devices may include: a support member which is disposed on one end of the light arm corresponding to the lamp module; a fixed link member which is rotatably coupled to the support member; and an insert link member which is disposed between the fixed link member and the support member.

In an embodiment of the disclosure, the support member may include: a body which accommodates a light source in the body; and a pair of supporting portions which extend from the body and are coupled to the fixed link member.

In an embodiment of the disclosure, the fixed link member may include: a receiving portion which accommodates a portion of the lamp module; and a pair of coupling portions which are disposed on respective sides of the receiving portion and coupled to the supporting portions via the insert link member.

In an embodiment of the disclosure, the lamp module may be configured to be replaced relative to the fixed link member.

In an embodiment of the disclosure, the fixed link member may further include a pair of legs which extend from the pair of coupling portions, respectively, and support portions of the lamp module.

In an embodiment of the disclosure, each of the angle adjustment devices may further include a fastening member which is connected to one of the pair of supporting portions and fixes or releases a position of the fixed link member relative to the support member.

In an embodiment of the disclosure, each of the angle adjustment devices may further include a guide which guides the angle of the lamp module relative to the light arm to be adjusted in a selected angle range.

### Mode for the Invention

Hereinafter, the following embodiments of the disclosure will be described in detail with reference to the accompanying drawings, in which identical or corresponding elements are designated by the same reference numerals and repeated descriptions thereof are omitted.

The embodiments may be variously modified, and specific embodiments will be illustrated in the accompanying drawings and described in detail in the detailed description. The effects and features of the embodiments and how to accomplish the same will be apparent with reference to the following detailed description together with the drawings. However, the embodiments are not limited to the embodiments disclosed below, but may be implemented in various forms.

In the following embodiments, the terms “first”, “second”, etc. are only used to distinguish one element from another element, rather than in a limiting sense.

In the following embodiments, the singular forms include the plural forms as well, unless the context clearly indicates otherwise.

In the following embodiments, the terms “include”, “have”, etc. indicate the existence of the features or elements described herein, but do not preclude the possibility of one or more other features or elements being added.

In the following embodiments, in a case where a portion, such as a unit, a region, or an element, is referred to as being above or on another portion, the portion may be directly above or on the other portion or an intervening portion, such as a unit, a region, or an element, may also be provided between the two portions.

In the following embodiments, the terms “connect”, “couple”, etc. do not necessarily imply a direct and/or fixed connection between two members unless the context clearly indicates otherwise, and do not exclude the possibility of other members being provided between the two members.

In the drawings, elements may be exaggerated or reduced in size for ease of explanation. For example, the sizes and thicknesses of the respective elements shown in the drawings are arbitrary for ease of explanation, and therefore the following embodiments are not necessarily limited thereto.

In the drawings, elements may be exaggerated or reduced in size for ease of explanation. For example, the sizes and thicknesses of the respective elements shown in the drawings are arbitrary for ease of explanation, and therefore the following embodiments are not necessarily limited thereto.

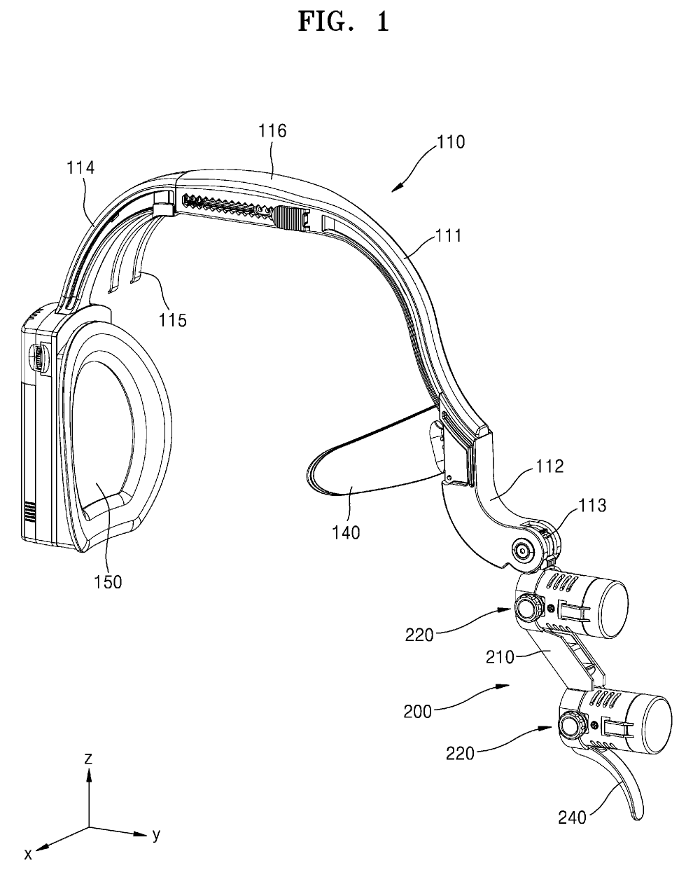

FIG. 1 is a perspective view illustrating a medical lighting device according to an embodiment of the disclosure.

Referring to FIG. 1, a medical lighting device 10 according to an embodiment of the disclosure includes a frame 110, a light source device 200, a forehead support 140, and an occiput support 150.

The medical lighting device 10 according to an embodiment of the disclosure improves the wearing comfort of a user by facilitating length adjustment for each element, and is characterized by including a plurality of light sources to ensure the field of view of the user. These features are explained in more detail as follows.

Referring again to FIG. 1, the frame 110 includes a main frame 111, a front frame 112, a rear frame 114, and a length adjustment portion 116.

The main frame 111 is provided along the front and top portions of the head of a wearer, extending from one end to form the front frame 112. The front frame 112 may be coupled to the light source device 200, the forehead support 140, and the like described later and perform the function of supporting the same.

The rear frame 114 is provided along the rear portion of the head of the wearer, and may be coupled to the occiput support 150 described later and serve to support the same. The rear frame 114 may also include an occiput support 115 separate from the occiput support 150. The occiput support 115 may perform the function of more stably supporting the occiput region of the user.

Here, the main frame 111 and the rear frame 114 may be provided as separate members to be coupled together, or at least portions of the main frame and the rear frame may be provided as one body.

The main frame 111 may include the length adjustment portion 116 for adjusting the length. The length adjustment portion 116 may function as a toggle switch, the main frame 111 may be movable or fixed relative to the rear frame 114, and thus whether length adjustment is possible may be determined. However, this is merely an example, and various mechanical configurations may be applied to the frame 110 as a length adjustment mechanism.

The forehead support 140 may be provided on a region of the main frame 111 corresponding to the forehead of the user, and may serve to support the forehead of the user. Such a forehead support 140 may be configured to move up and down relative to the frame 110. For example, the forehead support 140 may be configured to retract into and/or withdraw from the frame 110 so that the position of the forehead support may be adjusted according to the user. Here, various mechanical mechanisms for adjusting the position of the forehead support 140 may be applied.

The occiput support 150 is coupled to one end of the rear frame 114, and may serve to support the occiput of the user. Such an occiput support 150 may be configured to move vertically relative to the frame 110. For example, the occiput support 150 may be configured to retract into and/or withdraw from the frame 110 so that the position of the occiput support may be adjusted according to the user. Here, various mechanical mechanisms for adjusting the position of the occiput support 150 may be applied.

Although not shown in the figures, a power supply (not shown) may be further provided in the occiput support 150. For example, a power supply (not shown) for driving the light source device 200 and the like may be further provided in the occiput support 150.

The light source device 200 is coupled to one end of the front frame 112 extending from the main frame 111, and may include two or more lamp modules 220.

Here, the two or more lamp modules 220 may be provided in the vertical direction in a case where the user wears the medical lighting device 10, for example, in the Z-axis direction as shown in the figures.

The two or more lamp modules 220 may be arranged vertically in regions adjacent to both eyes of the user. For example, the two or more lamp modules 220 may be arranged in a direction substantially perpendicular to the straight line connecting the two eyes of the user.

Here, in a case where the medical lighting device is provided with only a single light source, shadows formed by the light source obstruct the field of view of a surgeon. To solve this problem, the medical lighting device 10 according to an embodiment of the disclosure features including the two or more lamp modules 220, which allow two light sources to interact and prevent shadow formation.

The lamp modules 220 may emit light toward a subject. Here, the lamp modules 220 may be implemented as any type of source device capable of generating light, and may be LEDs capable of emitting light in a specific wavelength band.

A handle 240 may be further provided on one side of the light source device 200 to adjust the position of the light source device 200. The light source device 200 may be rotatably connected to the frame 110 via one end of the front frame 112 and a hinge 113 in a direction parallel to the frame. For example, the angle and position of the light source device 200 relative to front frame 112 may be adjusted via the handle 240, and the position of the light source device 200 may be easily adjusted using this handle 240 even during surgery.

Hereinafter, the configuration and operation of the light source device 200 according to an embodiment of the disclosure will be described in detail.

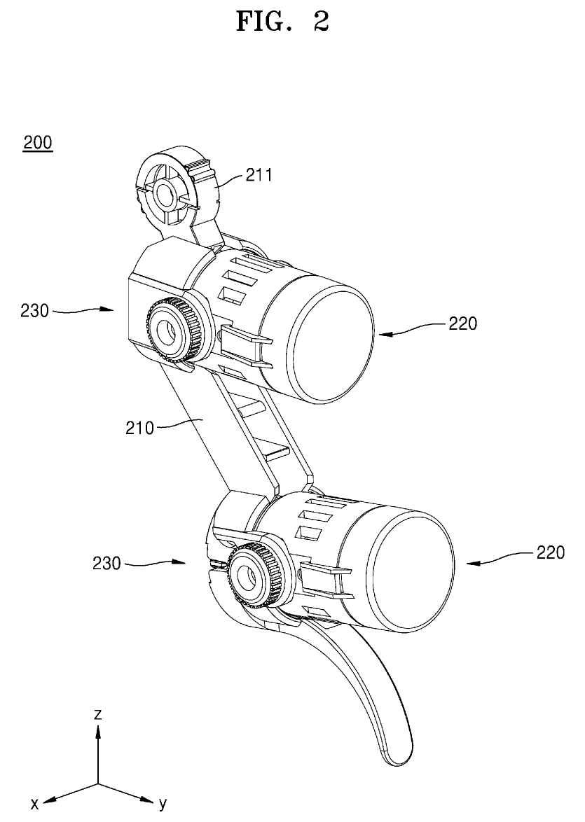

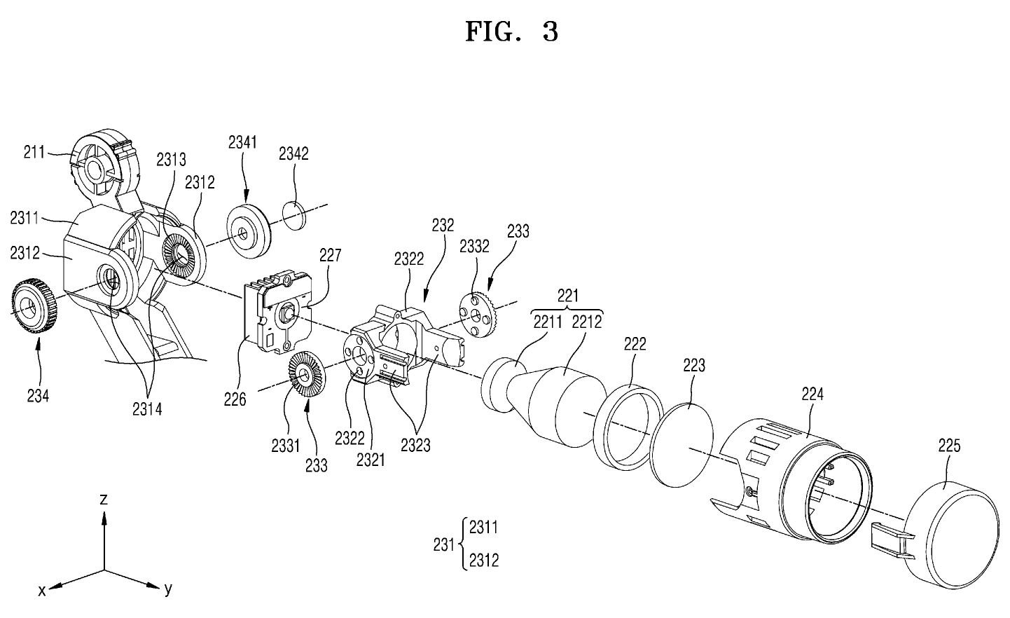

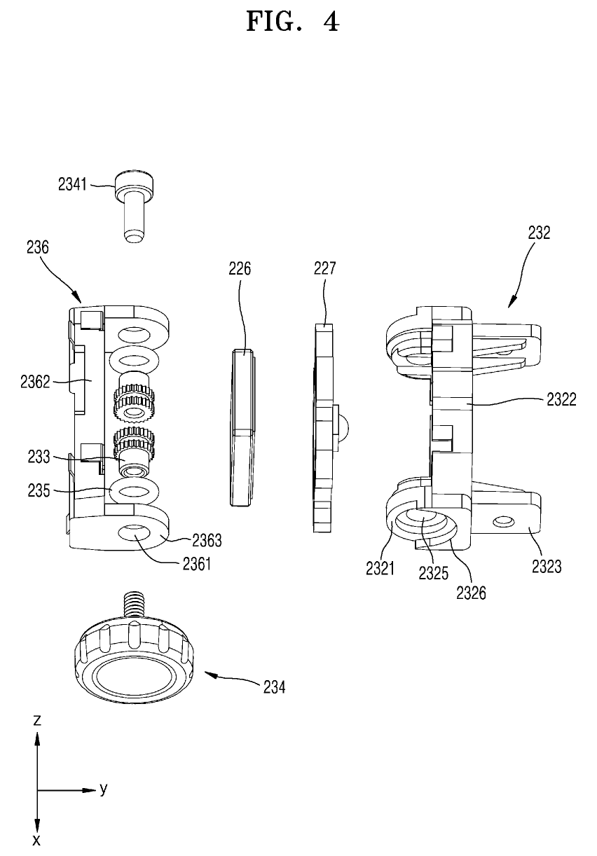

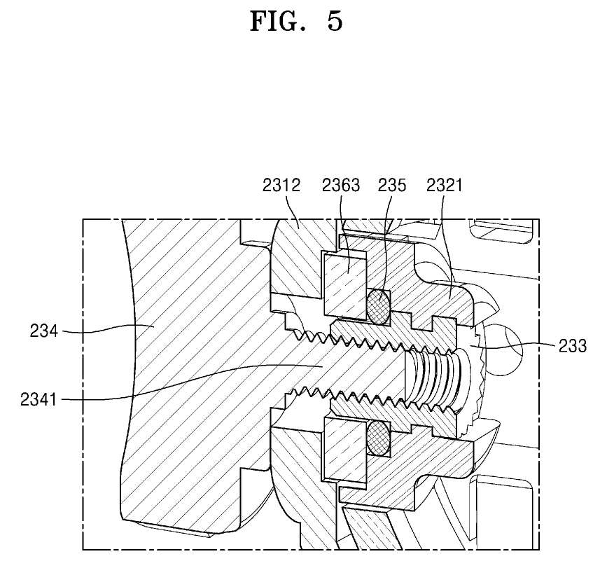

FIG. 2 is a perspective view only illustrating a light source device 200 in a medical lighting device 10 according to another embodiment of the disclosure. FIG. 3 is an exploded perspective view of the light source device 200 shown in FIG. 2, and FIG. 4 is an exploded perspective view of an angle adjustment device 230 of the light source device 200 shown in FIG. 3. FIG. 5 is a cross-sectional view illustrating a coupling structure between a fixed link member 232 and a light arm 210 in the medical lighting device 10 according to another embodiment of the disclosure.

Referring to FIGS. 1 to 3, in the medical lighting device 10 according to another embodiment of the disclosure, the light source device 200 includes the light arm 210, lamp modules 220, angle adjustment devices 230, a handle 240, and a frame connector 211.

The light arm 210 serves to support the plurality of lamp modules 220, and may be axially coupled to the hinge 113 of the front frame 112 due to the frame connector 211 provided on one end of the light arm. The handle 240 may be coupled to the other end.

The angle adjustment devices 230 may perform the function of connecting the light arm 210 and the plurality of lamp modules 220, so that the angle between the light arm and each of the lamp modules is adjustable.

In the medical lighting device 10 according to an embodiment of the disclosure, each of the angle adjustment devices 230 may include a support member 231, a fixed link member 232, and insert link members 233, and may further include a fastening member 234, O-rings 235, and a lamp holder 236.

Referring further to FIG. 4, the fixed link member 232 includes a receiving portion 2322 which accommodates a portion of the corresponding lamp module 220 and a pair of coupling portions 2321 which are disposed on respective sides of the receiving portion 2322 and are coupled to supporting portions 2312 via the insert link members 233.

Here, connectors 2363 of the lamp holder 236 may be disposed between the coupling portions 2321 and the supporting portions 2312. For example, in a case where the fixed link member 232 and the lamp holder 236 are coupled, the coupling portions 2321 may be disposed coaxially with the connectors 2363 and in the connectors 2363.

Each of the coupling portions 2321 may include a guide 2326 protruding in the thickness direction from one surface which is coupled to the corresponding connector 2363. In this case, the guide 2326 may have a shape which conforms to the shape of the connector 2363 and surrounds the connector 2363, as shown in FIG. 4. In adjustment of the angle of the fixed link member 232 relative to the lamp holder 236, the guide 2326 may perform the function of preventing rotation beyond a selected angle.

The coupling portions 2321 may have coupling holes 2325, respectively, which allow the coupling portions to be coupled to the fastening member 234 via the insert link members 233 and the O-rings 235.

The insert link members 233 may be implemented as insert nuts and may be inserted into and fixed in the coupling holes 2325 of the coupling portions 2321. The insert link members 233 may be coupled to the connectors 2363 with the O-rings 235 fitted on the insert link members.

The fastening member 234 may include a handle and a male thread. Here, the fastening member 234 may have a structure which functions as a bolt and engages with the corresponding insert link member 233 which functions as a nut.

Referring to FIG. 5, in the medical lighting device 10 according to another embodiment of the disclosure, the light arm 210, the lamp holder 236, and the fixed link member 232 may be fitted together by disposing the supporting portion 2312 of the light arm 210, the connector 2363 of the lamp holder 236, and the coupling portion 2321 of the fixed link member 232 between the fastening member 234 and the insert link member 233 and coupling the fastening member 234 and the insert link member 233 to each other.

Here, the O-rings 235 which are coupled to the insert link members 233 may be disposed between the coupling portions 2321 and the connectors 2363, and may be formed from a rubber material. As a result, the function of generating friction between the coupling portions 2321 and the connectors 2363, as well as adjusting the friction torque via the stepless angle adjustment, may be performed. For example, instead of being fixed at a selected angle, the lamp modules 220 may be fixed to remain at any desired angle.

The pressure applied to the O-rings 235 may be adjusted by controlling the degree of fastening of the fastening member 234 and the insert link members 233. This may set the lamp modules 220 to a state where the angle of the lamp modules is adjustable or to a state where the angle of the lamp modules is non-adjustable.

The lamp holder 236 may include connectors 2363 which extend from the body and be coupled to the coupling portions 2321 of the fixed link member 232, wherein each of the connectors 2363 may include a coupling hole 2361 into which the corresponding fastening member 234 may be inserted.

The connectors 2363 may be disposed between the coupling portions 2321 and the supporting portions 2312. For example, in a case where the fixed link member 232, the lamp holder 236, and the light arm 210 are coupled, the lamp holder 236 may accommodate the fixed link member 232, and the light arm 210 may accommodate the lamp holder 236. In this case, the connectors 2363 may be disposed coaxially with the coupling portions 2321 while being disposed outside the coupling portions 2321.

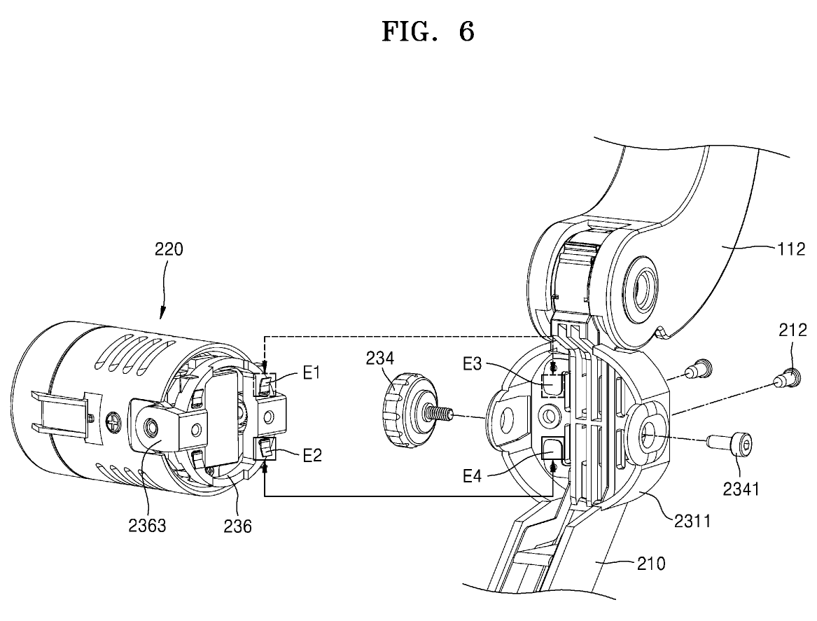

FIG. 6 is a view illustrating a disassembly/assembly structure and a power supply structure for the lamp modules 220 and the light arm 210 in the medical lighting device 10 according to an embodiment of the disclosure.

Referring to FIG. 6, the lamp holder 236 may further include contact terminals E1 and E2 for supplying power to the lamp modules 220. The contact terminals E1 and E2 may supply power to an LED light source 227 of the lamp module 220 by contacting the corresponding contact terminals E3 and E4 of the light arm 210. In the plurality of lamp modules 220 coupled to the light arm 210, the upper lamp module 220 and the lower lamp module 220 may have different terminal arrangements. For example, the contact terminals E1 and E2 of the upper lamp module 220 may be disposed on the left or right side of the lamp holder 236, while the contact terminals E1 and E2 of the lower lamp module 220 may be disposed on the top or bottom of the lamp holder 236.

The lamp holder 236 is coupled to the fixed link member 232 to form a semi-finished product of the lamp module 220, and enables the configuration of the lamp module 220 including the fixed link member 232 to be fastened to the light arm 210. For example, the lamp holder 236 may serve as a coupling medium between the light arm 210 and the lamp module 220. In an embodiment, in a case where the semi-finished product of the lamp module 220 is fastened to the light arm 210 in the light source device 200, the lamp holder 236 and the light arm 210 may be initially fixed using a rear screw 212, and the lamp module 220 and the light arm 210 may be secondarily fixed using the fastening member 234 and a side screw 2341.

In the medical lighting device 10 according to an embodiment of the disclosure, the angle adjustment devices 230 and the lamp modules may be configured as a single body to form the module as an integral component, thereby enabling quality verification at the component stage, facilitating repair and replacement, and allowing for easy post management without procedures such as wiring work.

Hereinafter, a medical lighting device 10 according to another embodiment of the disclosure will be described.

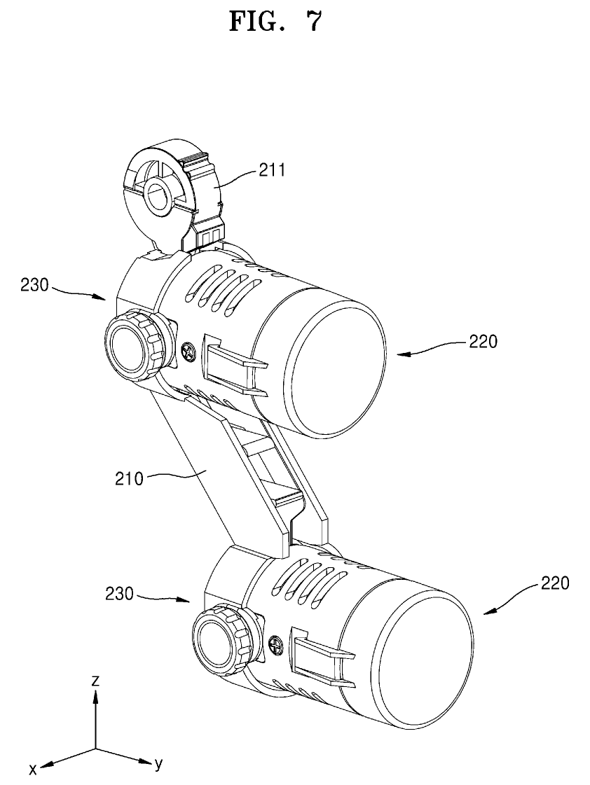

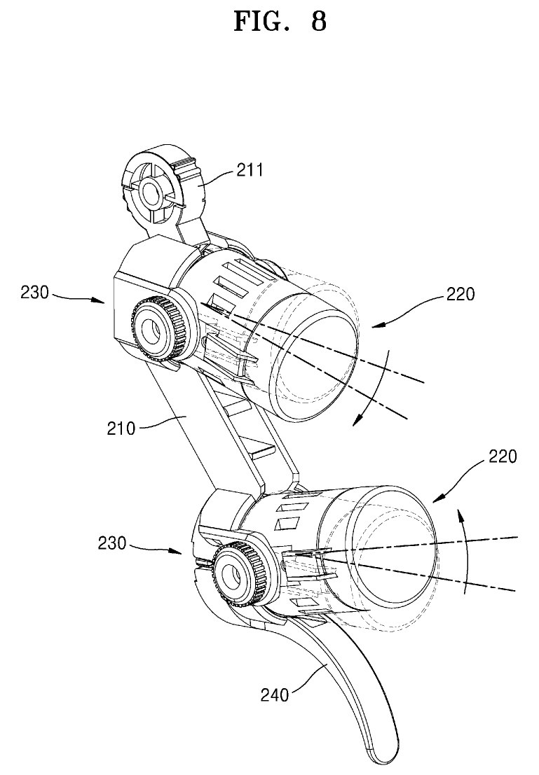

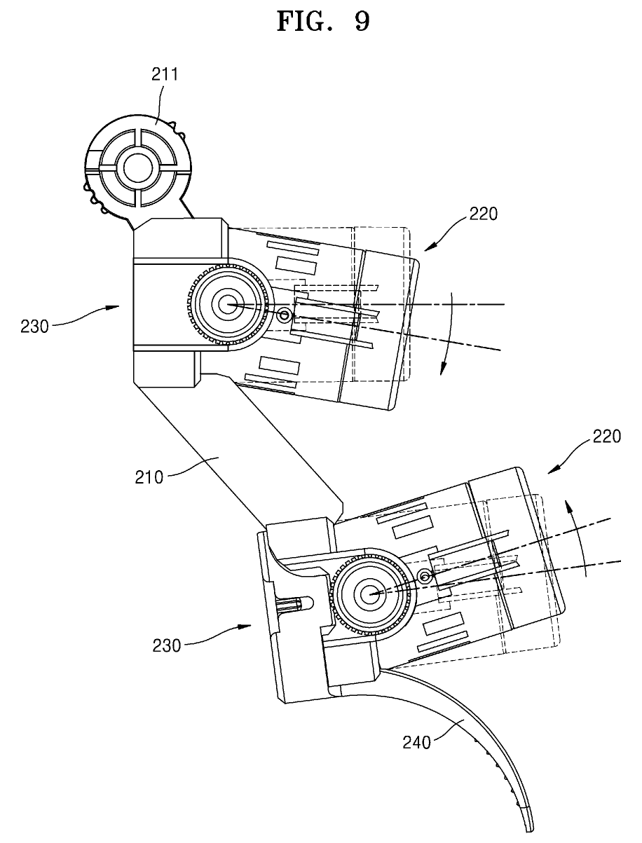

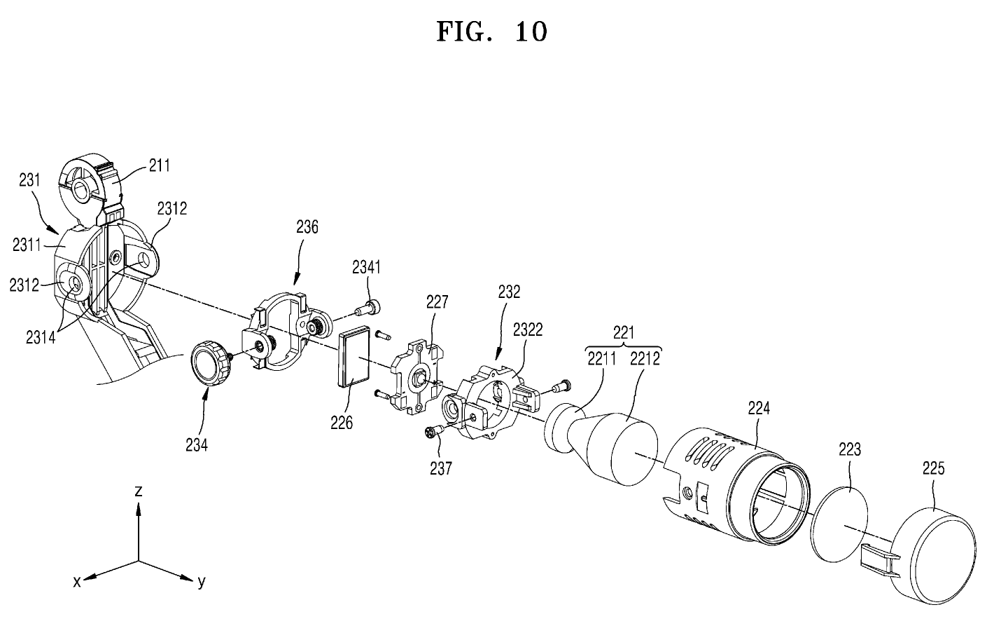

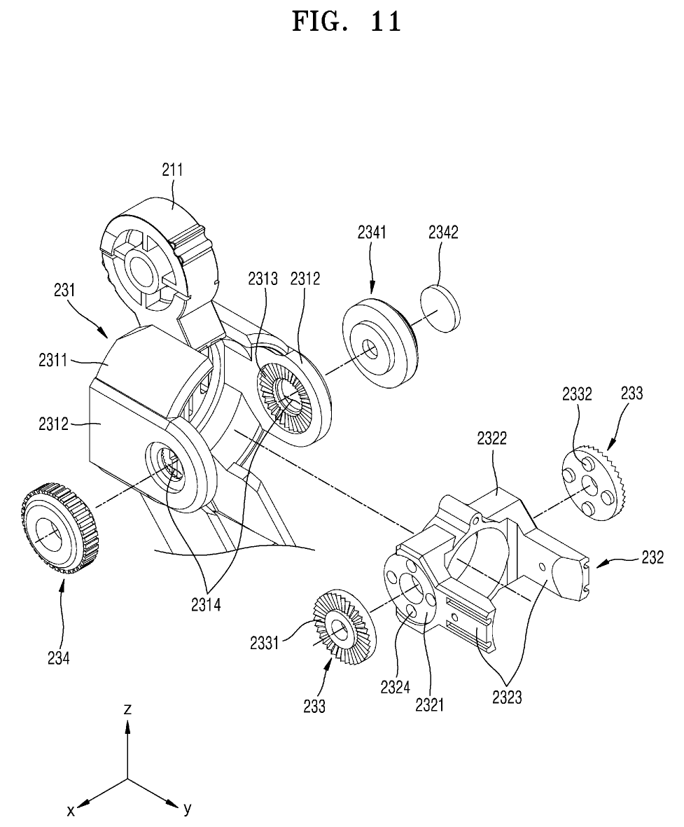

FIG. 7 is a perspective view only illustrating a light source device 200 in the medical lighting device 10 shown in FIG. 1. FIG. 8 is a perspective view illustrating the states of the respective lamp modules 220 in the light source device 200 shown in FIG. 7 before and after angle adjustment, and FIG. 9 is a side view illustrating the states of the respective lamp modules 220 in the light source device 200 shown in FIG. 7 before and after angle adjustment. FIG. 10 is an exploded perspective view illustrating the light source device 200 shown in FIG. 7, and FIG. 11 is an exploded perspective view illustrating the angle adjustment device 230 in the light source device 200 shown in FIG. 7.

Referring to FIGS. 7 to 11, in the medical lighting device 10 according to another embodiment of the disclosure, the light source device 200 includes a light arm 210, lamp modules 220, angle adjustment devices 230, a handle 240, and a frame connector 211.

Each of the angle adjustment devices 230 may include a support member 231 disposed on one end of the light arm 210 corresponding to the lamp module 220, a fixed link member 232 rotatably coupled to the support member 231, and insert link members 233 disposed between the fixed link member 232 and the support member 231. For example, the support member 231 may be provided on the top and the bottom of the light arm 210 as a portion capable of accommodating the plurality of lamp modules 220.

The support member 231 may include a body 2311 which accommodates a light source in the body and a pair of supporting portion 2312 which extend from the body 2311 and are coupled to the fixed link member 232. For example, the body 2311 may have an internal cavity configured to accommodate an assembly which includes the LED light source 227 and a heat sink 226 of the lamp module 220, and the supporting portion 2312 may be provided to extend from respective sides of the body 2311.

The supporting portion 2312 may have first toothed portions 2313 provided on surfaces facing the insert link members 233, and the insert link members 233 may have second toothed portions 2331 provided on surfaces corresponding to the supporting portion 2312.

Specifically, the first toothed portions 2313 may be provided on the inner surfaces which face each other at the ends of the supporting portion 2312 which extend from the respective sides of the body 2311. Each of the first toothed portions 2313 may have teeth which extend outward from the central portion of a circle having a selected diameter, resembling the shape of a crown gear. Similarly, each of the second toothed portions 2331 provided on the insert link members 233 may also have teeth provided in the shape of a crown gear like the first toothed portions 2313. However, the first toothed portions 2313 and the second toothed portions 2331 mesh with each other, but in a case where a selected or greater amount of force is applied, slip may occur, thereby allowing the second toothed portions 2331 to rotate in a staggered manner relative to the first toothed portions 2313.

A coupling hole 2314 may be provided in the central portion of each of the first toothed portions 2313. Each of the insert link members 233 may be provided with a protruding central portion which matches the size of the coupling hole 2314 and may pass through the coupling hole 2314.

Referring to FIGS. 10 and 11, each of the angle adjustment devices 230 may further include a fastening member 234 which is connected to one of the pair of supporting portion 2312 and fixes or releases the position of the fixed link member 232 relative to the support member 231.

The fastening member 234 may also be provided with a protruding central portion which matches the size of the coupling hole 2314, in which the central portion of the fastening member 234 which has passed through the coupling hole 2314 may be connected and coupled to the central portion of the insert link members 233.

One of the fastening members 234 may have anti-slip recesses provided on the outer surface to prevent slippage, and the other of the fastening members may have a smooth outer surface.

Referring again to FIGS. 7 to 11, the fixed link member 232 may include a receiving portion 2322 which accommodates a portion of the lamp module 220 and a pair of coupling portions 2321 which are disposed on respective sides of the receiving portion 2322 and coupled to the supporting portion 2312 via the insert link members 233. The pair of coupling portions 2321 may be provided with a plurality of recesses 2324 which fixedly engage with the insert link members 233.

Specifically, coupling portions 2321 are present on respective sides of the fixed link member 232 to be coupled to the insert link members 233, and the plurality of recesses 2324 provided in the coupling portions 2321 may engage with protrusions 2332 of the insert link members 233 to fit the insert link members 233 to the fixed link member 232. An assembly which includes the insert link member 233, the fixed link member 232, and the insert link member 233 is positioned between the pair of supporting portion 2312, and the coupling holes 2314 of the supporting portion 2312 and the coupling portions 2321 of the fixed link member 232 are positioned on the same axis.

The receiving portion 2322 of the fixed link member 232 serves to accommodate and fix the components of the lamp modules 220. For example, the receiving portion may accommodate and fix the assembly of the LED light source 227 and the heat sink 226, and may accommodate and hold a lens 221. Specifically, the receiving portion 2322 may have a hollow central portion, into which one end 2211 of the lens 221 may be inserted to fit the lens and the receiving portion together.

The fixed link member 232 may further include a pair of legs 2323 which extend from the pair of coupling portions 2321, respectively, to support portions of the lamp module 220. The pair of legs 2323 may accommodate and fix the lens 221, a light cover 224, and the like.

In the medical lighting device 10 according to another embodiment of the disclosure, the lamp modules 220 may be configured to be replaced relative to the fixed link member 232. For example, referring to FIG. 10, the lamp modules 220 may include a lens 221, a fixed ring 222, a cover glass 223, a light cover 224, a light filter 225, and the like, wherein the components which are coupled to the receiving portion 2322 and the legs 2323 of the fixed link member 232 may be detached from the fixed link member 232.

In conventional medical lighting devices, it is impossible to disassemble and reassemble the light source device because the components corresponding to the light arm were integrated with the lamp modules, but in the medical lighting device 10 according to another embodiment of the disclosure, it is possible to disassemble and reassemble some components of the lamp modules 220, thereby enabling replacement of the lens 221 and the like.

According to another embodiment of the disclosure, the lamp modules 220 may be connected to the light arm 210 in an angle-adjustable manner.

Referring again to FIGS. 8 and 9, the states before and after the angles of the lamp modules 220 are narrowed relative to the light arm 210 may be observed. The angles of the lamp modules 220 relative to the light arm 210 may be adjusted using the angle adjustment devices 230.

For example, the lamp modules 220 are coupled to the fixed link member 232 to form an assembly, and because the fixed link member 232 is coupled to the supporting portion 2312 by the insert link members 233, the angles of the lamp modules 220 may be adjusted vertically on the coupling portions 2321 which serve as axes.

Although not shown, each of the angle adjustment devices 230 may further include a guide (not shown) which guides the angle of the corresponding lamp module relative to the light arm 210 to be adjusted in a selected angle range. Each of the angle adjustment devices 230 may include a protrusion which is provided on the supporting portion 2312 and is configured to prevent the lamp module 220 from rotating beyond the selected angle or a range adjustment guide which is provided on the first toothed portion 2313 or the second toothed portion 2331.

In an embodiment, in a case where the user adjusts the angle of one lamp module 220, the guide (not shown) may perform the function of enabling the angle of the other lamp module 220 to be also adjusted. Consequently, the medical lighting device 10 allows the user to conveniently focus the two lamp modules 220 at a desired position by only adjusting the angle of one lamp module 220.

In another example, in a case where the user adjusts the angle of one lamp module 220, the guide (not shown) may perform the function of limiting the available angle adjustment range of the other lamp module 220 depending on the adjusted angle.

Conventional medical headlamps have the problem that the focal length at which light emitted from each lamp module converges is fixed and thus the distance between each headlamp and a surgical site is also fixed, thereby making it difficult to use such medical headlamps in various surgical environments. In contrast, the medical lighting device 10 according to the embodiments of the disclosure may adjust the focal length by controlling the angle of each lamp module 220 of the light source device 200, thereby adjusting the distance from a surgical site to be closer or farther depending on the surgical environment of the user.

As described above, the medical lighting device according to the embodiments of the disclosure may provide a medical lighting device which includes a plurality of light sources to ensure the field of view of a user and facilitates adjustment of the angles of the light sources and enables disassembly and reassembly of respective lamp modules of a light source device, thereby improving the convenience of use.

The disclosure has been described hereinabove with reference to the example embodiments. A person having ordinary knowledge in the art to which the disclosure pertains will appreciate that various modifications are possible without departing from the essential features of the disclosure. The above-disclosed methods are therefore to be considered in an illustrative, not a limiting, perspective. The scope of the disclosure is to be construed as being defined by the claims, but by the foregoing description, and to encompass all equivalent differences.

### Industrial Applicability

Embodiments of the disclosure relate to a medical lighting device. The disclosure may be applied to a medical lighting device which includes a plurality of light sources to ensure the field of view of a user and facilitates adjustment of the angles of the light sources and enables disassembly and reassembly of respective lamp modules of a light source device, thereby improving the convenience of use.

1. A medical lighting device wearable on a head of a user comprising:
a frame provided along front and top portions of a head of a user; and
a light source device coupled to one end of the frame to be rotatable relative to the frame,
wherein the light source device comprises:
a plurality of lamp modules which are arranged in a direction substantially perpendicular to a straight line connecting two eyes of the user in a case where the user wears the medical lighting device;
a light arm comprising a first contact terminal, wherein the light arm supports the plurality of lamp modules and is rotatably coupled to the frame; and
angle adjustment devices, each of which connects the light arm and a corresponding lamp module of the plurality of lamp modules so that an angle of the lamp module is adjustable relative to the light arm,
wherein each of the angle adjustment devices comprises:
a support member comprising a body and a supporting portion which extends from the body, wherein the support member is disposed on one end of the light arm corresponding to the lamp module;
a fixed link member comprising a receiving portion which accommodates the lamp module and a coupling portion which is coupled to the supporting portion, wherein the fixed link member is rotatably coupled to the support member;
a lamp holder comprising a second contact terminal which supplies power to the lamp module by contacting the first contact terminal and a connector which is disposed between the coupling portion and the supporting portion, wherein the lamp holder accommodates the fixed link member;
a fastening member which is disposed outside the supporting portion; and
an insert link member which is inserted into the coupling portion and coupled to the fastening member, wherein
the supporting portion, the connector, and the coupling portion are fastened using the fastening member and the insert link member, thereby causing the fixed link member to be rotatably coupled to the lamp holder and the lamp holder to be fixedly coupled to the support member, and
the angles of the plurality of lamp modules are adjusted, thereby causing a focal length at which light emitted from the lamp modules converges to be adjustable.

2. The medical lighting device of claim 1,
wherein the lamp module is configured to be replaced relative to the fixed link member.

3. The medical lighting device of claim 1,
wherein the fixed link member comprises:
a pair of coupling portions; and
a pair of legs which extend from the pair of coupling portions,
respectively, and support portions of the lamp module.

4. The medical lighting device of claim 1,
wherein a degree of fastening of each of the fastening member and the insert link member is adjusted, thereby setting the lamp module to a state where the angle of the lamp module is adjustable or to a state where the angle of the lamp modules is non-adjustable.

5. The medical lighting device of claim 1,
wherein each of the angle adjustment devices further comprises a guide which guides the angle of the lamp module relative to the light arm to be adjusted in a selected angle range.
